(19) United States
(12) Patent Application Publication
(10) Pub. No.: US 2026/0294413 A1
(43) Pub. Date: Oct. 1, 2026

(54) PRE-TENSIONING PULL WIRES IN ROBOTIC SURGICAL INSTRUMENTS

(71) Applicant: Auris Health, Inc., Santa Clara, CA (US)

(72) Inventors: Robert Mason GLIDDEN, IV, Emerald Hills, CA (US); Boney Charles MEJIA, Mountain View, CA (US)

(73) Assignee: Auris Health, Inc., Santa Clara, CA (US)

(21) Appl. No.: 19/093,099

(22) Filed: Mar. 27, 2025

(51) Int. Cl. A61B 17/00

(52) U.S. Cl. CPC A61B 17/00234; A61B 2017/00327

(57) ABSTRACT

A surgical system is disclosed that includes a housing, a scope extending from the housing, first and second wires operably coupled to the scope and extending into the housing, a pulley rotatably mounted within the housing and defining a slot extending between first and second supports of the pulley, and a wire anchor received within the slot and configured to receive the first and second wires, wherein joining the wire anchor to the first and second wires operably couples the first and second wires to the pulley.

### BACKGROUND

The present disclosure relates to robotic surgical systems and, more particularly, to systems and methods for pre-tensioning pull wires in robotic surgical instruments and thereby helping to eliminate backlash.

In the field of robotic surgery, precision and control are paramount for ensuring successful surgical outcomes. Robotic surgical instruments are designed to mimic the movements of a surgeon’s hand with high accuracy and repeatability. However, one significant challenge in the development of such instruments is the phenomenon of backlash, which refers to the slight movement or "play" in mechanical systems when changing the direction of motion. This backlash can lead to inaccuracies in the instrument’s position or force transmission, potentially compromising the precision of the surgical procedure.

Backlash typically occurs in mechanical systems that rely on gears, motors, and other drive mechanisms. In robotic surgical instruments, backlash can manifest when a servo motor, gear train, or transmission component shifts direction. The inherent "slack" or "gap" in these interconnected components can delay or distort the transmission of motion, causing the surgical tool to deviate from the intended trajectory. This effect can be particularly problematic during high-precision procedures, such as microsurgery, where even minute deviations from the planned path can result in significant complications.

Minimizing backlash is crucial for ensuring that robotic systems perform with the necessary levels of accuracy and reliability. The presence of backlash in robotic surgical instruments can lead to several undesirable outcomes, including imprecise movements, erratic tool positioning, difficulty in maintaining consistent pressure, and reduced overall performance. In critical surgical procedures, these issues could result in damage to sensitive tissues or even patient harm.

Accordingly, systems and methods for eliminating backlash in robotic surgical instruments are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates an example medical system that can incorporate the principles of the present disclosure.

FIG. 2 illustrates medical system components that may be implemented in the medical system of FIG. 1.

FIG. 3 shows an exploded view of an instrument manipulator assembly of FIG. 1 that includes a surgical instrument, an adapter, and a robotic arm.

FIG. 4 is a cross-sectional view of the scope as taken along the lines indicated in FIG. 3.

FIG. 5 is an isometric view of the interior of the surgical instrument of FIG. 3, in accordance with at least one aspect of the present disclosure.

FIG. 6 is a partial, top-down view of the surgical instrument of FIG. 5, in accordance with at least one aspect of the present disclosure.

FIG. 7 is a partial, rear-isometric view of the surgical instrument of FIG. 5, in accordance with at least one aspect of the present disclosure.

FIG. 8 illustrates tensioning pull wires of the surgical instrument of FIG. 5, in accordance with at least one aspect of the present disclosure.

FIG. 9 illustrates joining a wire anchoring member to a pulley of the surgical instrument of FIG. 5, in accordance with at least one aspect of the present disclosure.

FIG. 10 is an isometric view of an improved intermediate rotary driver of the adapter of FIG. 3, in accordance with at least one aspect of the present disclosure.

FIG. 11 is the intermediate rotary driver of FIG. 10 coupled to a capstan of the surgical instrument of FIG. 3, in accordance with at least one aspect of the present disclosure.

FIG. 12 is a cross-sectional view of FIG. 11, in accordance with at least one aspect of the present disclosure.

FIG. 13 is the intermediate rotary driver of FIG. 10 coupled to a rotary output of the robotic arm of FIG. 3, in accordance with at least one aspect of the present disclosure.

### DETAILED DESCRIPTION

The present disclosure relates to surgical systems and, more particularly, to systems and methods for securing components within a surgical instrument housing.

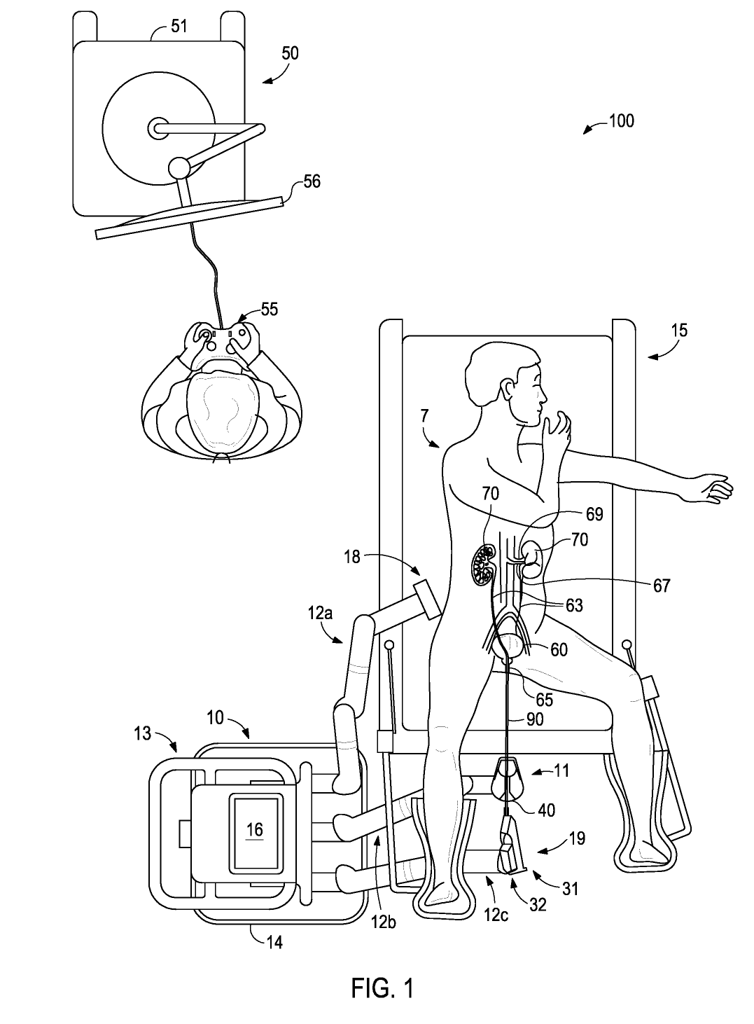

FIG. 1 illustrates an example medical system 100 that may incorporate aspects of the present disclosure. In some applications, the medical system 100 may be used for endoscopic (e.g., ureteroscopic) procedures. Kidney stone treatment can benefit from the assistance of certain robotic technologies/devices, such as may be similar to those shown in FIG. 1 and described in detail below. Additional information regarding medical system 100 is provided in U.S. Pat. No. 12,097,079, titled “STUCK INSTRUMENT MANAGEMENT”, which issued on Sep. 24, 2024, and which is hereby incorporated by reference in its entirety herein. Although the system 100 of FIG. 1 is presented in the context of a ureteroscopic procedure, it should be understood that the principles disclosed herein may be implemented in any type of endoscopic and/or percutaneous procedure.

The medical system 100 includes a robotic system 10 (e.g., mobile robotic cart) configured to engage with and/or control a medical instrument 40 (e.g., ureteroscope) to perform a direct-entry procedure on a patient 7. The term “direct-entry” is used herein according to its broad and ordinary meaning and may refer to any entry of instrumentation through a natural or artificial opening in a patient's body. For example, with reference to FIG. 1, the direct entry of the scope 40 into the urinary tract of the patient 7 may be made via the urethra 65. It should be understood that the direct-entry instrument 40 may be any type of medical instrument, including an endoscope (such as a ureteroscope), catheter (such as a steerable or non-steerable catheter), nephroscopes, laparoscope, or other type of medical instrument.

The medical system 100 includes a control system 50 configured to interface with the robotic system 10, provide information regarding the procedure, and/or perform a variety of other operations. For example, the control system 50 includes one or more display(s) 56 configured to present certain information to assist the physician 5 and/or other technician(s) or individual(s). The medical system 100 can include a table 15 configured to hold the patient 7. The system 100 may further include an electromagnetic (EM) field generator 18, which may be held by one or more of the robotic arms 12a of the robotic system 10 or may be a stand-alone device.

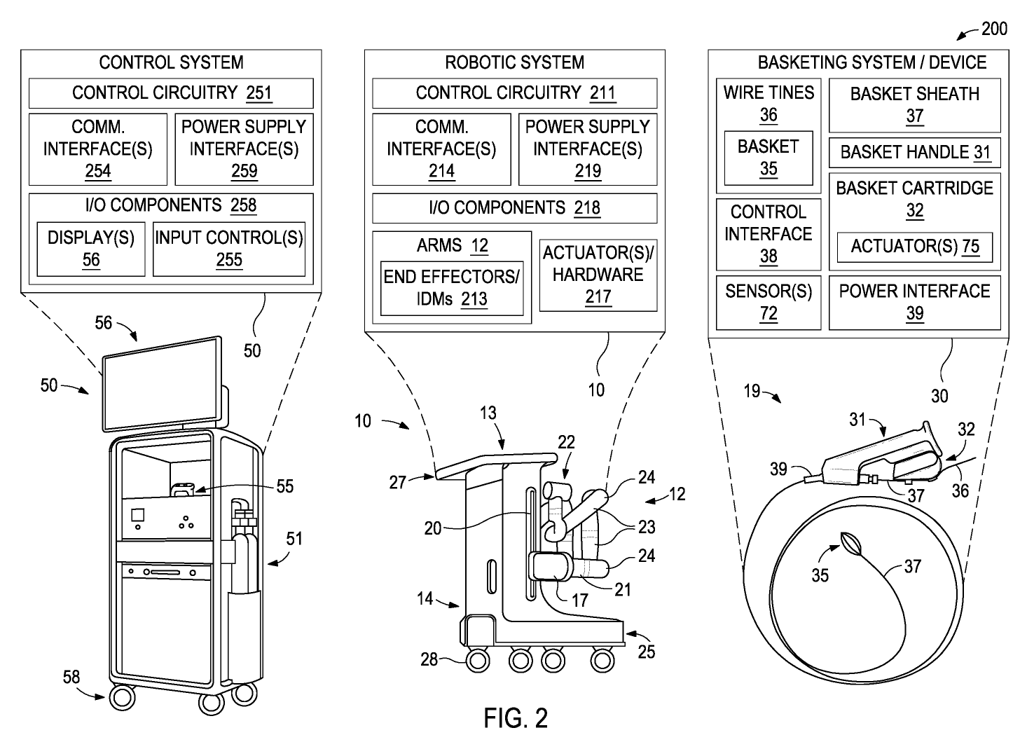

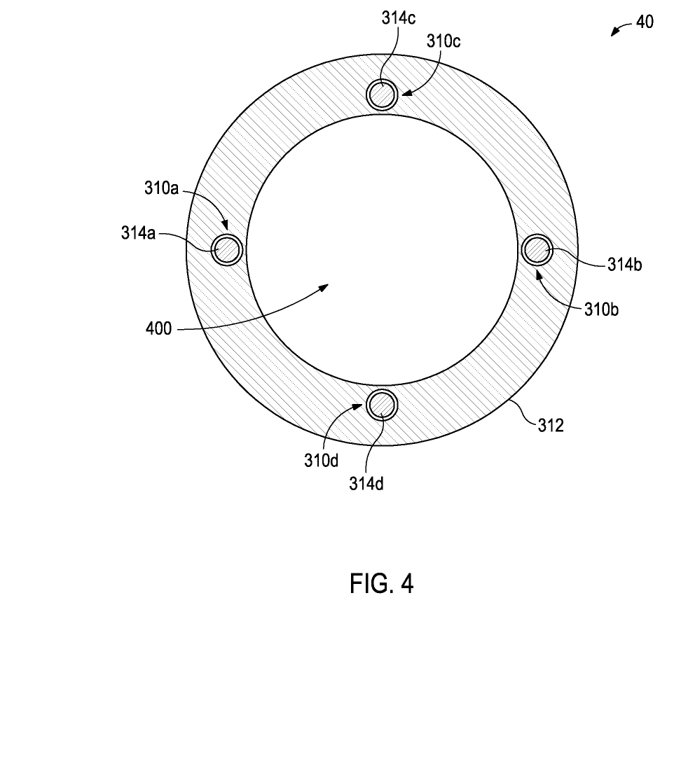

In an example use case, if the patient 7 has a kidney stone located in the kidney 70, the physician may execute a procedure to remove the stone through the urinary tract (63, 60, 65). The physician 5 can interact with the control system 50 and/or the robotic system 10 to cause/control the robotic system 10 to advance and navigate the medical instrument 40 (e.g., a scope) from the urethra 65, through the bladder 60, up the ureter 63, and into the kidney 70 where the stone is located. The physician 5 can further interact with the control system 50 and/or the robotic system 10 to cause/control the advancement of a basketing device 30 (FIG. 2) through a working channel 400 (FIG. 4) of the instrument 40, wherein the basketing device 30 (FIG. 2) is configured to facilitate capture and removal of a kidney stone. The control system 50 can provide information via the display(s) 56 that is associated with the medical instrument 40, such as real-time endoscopic images captured therewith, and/or other instruments of the system 100, to assist the physician 5 in navigating/controlling such instrumentation.

The medical instrument 40 (e.g., scope, directly-entry instrument, etc.) can be advanced into the kidney 70 through the urinary tract. Specifically, a ureteral access sheath 90 may be disposed within the urinary tract and advanced to an area near the kidney 70. The medical instrument 40 may be passed through the ureteral access sheath 90 to gain access to the internal anatomy of the kidney 70. Once at the site of the kidney stone, the medical instrument 40 can be used to channel/direct the basketing device 30 (FIG. 2) to the target location. Once the stone has been captured in the distal basket portion 35 (FIG. 2) of the basketing device 30, the ureteral access path may be used to extract the kidney stone from the patient 7.

The scope-type instruments disclosed herein, such as the scope 40 of the system 100, can be configured to navigate within the human anatomy, such as within a natural orifice or lumen of the human anatomy. The terms “scope” and “endoscope” are used herein according to their broad and ordinary meanings, and may refer to any type of elongate medical instrument having image generating, viewing, and/or capturing functionality and configured to be introduced into any type of organ, cavity, lumen, chamber, or space of a body. A scope can include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise a rigid or flexible tube, and may be dimensioned to be passed within an outer sheath, catheter, introducer, or other lumen-type device, or may be used without such devices.

Referring to FIGS. 1 and 2, the control system 50 can be configured to provide various functionality to assist in performing a medical procedure. In some embodiments, the control system 50 can be coupled to the robotic system 10 and operate in cooperation therewith to perform a medical procedure on the patient 7. For example, the control system 50 can communicate with the robotic system 10 via a wireless or wired connection (e.g., to control the robotic system 10). Further, in some embodiments, the control system 50 can communicate with the robotic system 10 to receive position data therefrom relating to the position of the distal end of the scope 40, access sheath 90, or basketing device 30. Such positional data relating to the position of the scope 40, access sheath 90, or basketing device 30 may be derived using one or more electromagnetic sensors associated with the respective components. In some embodiments, the control system 50 can communicate with the EM field generator 18 to control generation of an EM field in an area around the patient 7.

With particular reference to FIG. 2, the robotic system 10 can be configured to at least partly facilitate execution of a medical procedure. The robotic system 10 can include one or more robotic arms 12 (12a, 12b, 12c) configured to engage with and/or control, for example, the scope 40 and/or the basketing system 30 to perform one or more aspects of a procedure. As shown, each robotic arm 12 can include multiple arm segments 23 coupled to joints 24, which can provide multiple degrees of movement/freedom. As shown in FIG. 1, the robotic system 10 is positioned proximate to the patient's legs and the robotic arms 12 are actuated to engage with and position the scope 40 for access into an access opening, such as the urethra 65 of the patient 7. When the robotic system 10 is properly positioned, the scope 40 can be inserted into the patient 7 robotically using the robotic arms 12b, manually by the physician 5, or a combination thereof. A scope-driver instrument coupling or “scope driver” 11 (i.e., instrument device manipulator (IDM)) can be attached to the distal portion of one of the arms 12b to facilitate robotic control/advancement of the scope 40. Another 12c of the arms has associated therewith an instrument coupling/manipulator 19 configured to facilitate advancement and operation of the basketing device 30. The scope 40 includes one or more working channels 400 (FIG. 4) through which additional tools, such as lithotripters, basketing devices, forceps, etc., can be introduced into the treatment site.

The robotic system 10 can be coupled to any component of the medical system 100, such as to the control system 50, the table 15, the EM field generator 18, the scope 40, the basketing system 30, and/or any type of percutaneous-access instrument (e.g., needle, catheter, nephroscope, etc.). In some embodiments, the robotic system 10 is communicatively coupled to the control system 50. For example, the robotic system 10 may be configured to receive control signals from the control system 50 to perform certain operations, such as to position one or more of the robotic arms 12 in a particular manner, manipulate the scope 40, manipulate the basketing system 30, and so on. In response, the robotic system 10 can control, using certain control circuitry 211, actuators 217, and/or other components of the robotic system 10, a component of the robotic system 10 to perform the operations. In some embodiments, the robotic system 10 and/or control system 50 is/are configured to receive images and/or image data from the scope 40 representing internal anatomy of the patient 7, namely the urinary system with respect to the particular depiction of FIG. 1, and/or display images based thereon.

With reference to FIG. 2, the robotic system 10 generally includes an elongated support structure 14 (also referred to as a “column”), a robotic system base 25, and a console 13 at the top of the column 14. The column 14 includes one or more arm supports 17 (also referred to as a “carriage”) for supporting the deployment of the one or more robotic arms 12 (three shown in FIG. 2). The arm support 17 includes individually-configurable arm mounts that rotate along a perpendicular axis to adjust the base of the robotic arms 12 for desired positioning relative to the patient.

The arm support 17 is configured to vertically translate along the column 14, and can be connected to the column 14 through slots 20 that are positioned on opposite sides of the column 14 to guide the vertical translation of the arm support 17. The slot 20 contains a vertical translation interface to position and hold the arm support 17 at various vertical heights relative to the robotic system base 25. Vertical translation of the arm support 17 allows the robotic system 10 to adjust the reach of the robotic arms 12 to meet a variety of table heights, patient sizes, and physician preferences. Similarly, the individually-configurable arm mounts on the arm support 17 can allow the robotic arm base 21 of robotic arms 12 to be angled in a variety of configurations.

The robotic arms 12 include robotic arm bases 21 and end effectors 22, separated by a series of linking arm segments 23 that are connected by a series of joints 24, each joint including one or more independent actuators 217. Each actuator 217 comprises an independently-controllable motor. Each independently-controllable joint 24 can provide or represent an independent degree of freedom available to the robotic arm. The robotic system base 25 can include wheel-shaped casters 28 that allow for the robotic system to easily move around the operating room prior to a procedure.

Positioned at the upper end of column 14, the console 13 can provide both a user interface for receiving user input and a display screen 16 (or a dual-purpose device such as, for example, a touchscreen) to provide the physician/user with both pre-operative and intra-operative data. As shown, the console 13 can also include a handle 27 to assist with maneuvering and stabilizing robotic system 10.

The end effector 213 of each robotic arm 12 includes, or is configured to have coupled thereto, an instrument device manipulator (IDM), which is attached using a mechanism changer interface (MCI). In some embodiments, the IDM can be removed and replaced with a different type of IDM, for example, a first type 11 of IDM manipulates an endoscope, while a second type 19 of IDM manipulates a basketing device. Another type of IDM is configured to hold an electromagnetic field generator 18. An MCI can provide power and control interfaces. For example, the interfaces can include connectors to transfer pneumatic pressure, electrical power, electrical signals, and/or optical signals from the robotic arm 12 to the IDM. The IDMs 213 may be configured to manipulate medical instruments (e.g., surgical tools/instruments), such as the scope 40, using techniques including, for example, direct drives, harmonic drives, geared drives, belts and pulleys, magnetic drives, and the like. In some embodiments, the medical device manipulators 213 can be attached to respective ones of the robotic arms 212, wherein the robotic arms 212 are configured to insert or retract the respective coupled medical instruments into or out of the treatment site.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211/251 may be entirely locally maintained/disposed or may be remotely located at least in part (e.g., communicatively coupled indirectly via a local area network and/or a wide area network).

With further reference to FIG. 2, the control system 50 can include various I/O components 258 configured to assist the physician 5 or others in performing a medical procedure. For example, the input/output (I/O) components 258 can be configured to allow for user input to control/navigate the scope 40 and/or basketing system within the patient 7. In some embodiments, for example, the physician 5 can provide input to the control system 50 and/or robotic system 10, wherein in response to such input, control signals can be sent to the robotic system 10 to manipulate the scope 40 and/or catheter basketing system 30. The control system 50 can include one or more display devices 56 to provide various information regarding a procedure.

To facilitate the functionality of the control system 50, the control system can include various components or “subsystems”. For example, the control system 50 can include the control electronics/circuitry 251, as well as one or more power supplies/supply interfaces 259, pneumatic devices, optical sources, actuators, data storage devices, and/or communication interfaces 254. In some embodiments, the control system 50 is movable, while in other embodiments, the control system 50 is a substantially stationary system.

The control system 50, basketing system 30, and/or robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore.

The basketing system 30 includes various hardware and control components. For example, as shown in FIG. 2, the basketing system 30 can include a basket 35 formed of one or more wire tines 36, such as four wire tines disposed within a basketing sheath 37 over a length thereof, wherein the tines project from a distal end of the sheath 37 to form the basket 35. The tines 36 further extend from the proximal end of the sheath 37. The tines 36 may be configured to be slidable within the basketing sheath 37, subject to some amount of frictional resistance. The tines 36 and the sheath 37 can be coupled to respective actuators 75 of a basket cartridge component 32. The basket cartridge 32 may be physically and/or communicatively coupled to a handle portion/component 31 of the basketing system 30. The handle component 31 can be configured to be used to assist in basketing control either manually or through robotic control.

The basketing system 30 can be powered through a power interface 39 and/or controlled through a control interface 38, each or both of which may interface with a robotic arm/component of the robotic system 10. The basketing system 30 further includes one or more sensors 72, such as pressure and/or other force-reading sensors, which are configured to generate signals indicating forces experienced at/by one or more of the actuators 75 and/or other couplings of the basketing system 30. In some embodiments, the sensor(s) 72 include one or more sensors configured to directly measure forces are at or near the basket portion 35 of the tines 36. For example, a force sensor on the tip of the basket 35 and/or at a tip of an access sheath through which the basketing device 30 accesses the target anatomy can be used to directly detect forces on the basket 35 that result from the basket 35 becoming stuck on anatomy or on an opening at an end of the access sheath.

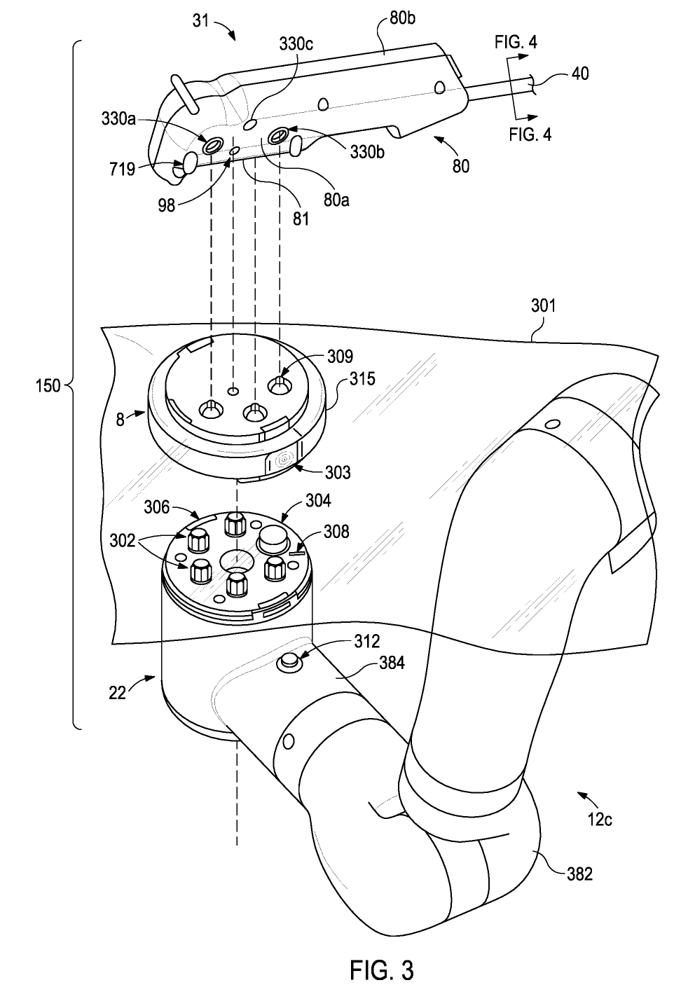

FIG. 3 shows an exploded view of an instrument device manipulator assembly 150 associated with the robotic arm 12c in accordance with one or more embodiments. The instrument device manipulator assembly 150 includes an end effector 22 associated with a distal end of the robotic arm 12c. The instrument manipulator assembly 150 further includes a handle 31 of a shaft-type instrument or instrument coupling/manipulator 19. The instrument handle 31 can incorporate mechanical (and/or electrical) means for rolling/rotating a shaft component associated therewith, such as an endoscope or other shaft-type instrument.

In some embodiments, the instrument device manipulator assembly 150 further includes an adapter component or “adapter” 8 that is mountable to the end effector 22 and configured to provide a drive interface between the end effector 22 and the instrument handle 31. The adapter 8 and/or the instrument handle 31 may be removable or detachable from the robotic arm 12c and may be devoid of any electro-mechanical components, such as motors, in some embodiments. This dichotomy may be driven by the need to sterilize medical instruments used in medical procedures and the inability to adequately sterilize expensive capital equipment due to their intricate mechanical assemblies and sensitive electronics. Accordingly, the instrument handle 31 and/or adapter 8 may be designed to be detached, removed, and interchanged from the end effector 22 (and thus the system) for individual sterilization or disposal. In contrast, the end effector 22 need not be changed or sterilized in some cases and may be draped (e.g., using drape 301) for protection.

In some embodiments, the adapter 8 can include connectors to transfer pneumatic pressure, electrical power, electrical signals, and/or optical signals from the robotic arm 12c and/or end effector 22 to the instrument handle 31. The robotic arm 12c can advance/insert or retract the coupled instrument handle 31 into or out of the treatment site. In some embodiments, the instrument handle 31 can be removed and replaced with a different type of instrument.

The end effector 22 of the robotic arm 12c can include various components/elements configured to connect to and/or align with components of the adapter 8, instrument handle 31, and/or scope 40. For example, the end effector 22 can include rotary drive outputs 302 (e.g., drive splines, gears, or rotatable disks with engagement features) to control/articulate a surgical instrument (e.g., the instrument handle 31), a reader 304 to read data from a surgical instrument (e.g., radio-frequency identification (RFID) reader to read a serial number from a surgical instrument), one or more fasteners 306 to attach the instrument handle 31 and/or adapter 8 to the end effector 22, marker(s) 308 to align with an instrument that is manually attached to a patient (e.g., access sheath) and/or to define a front surface of the device manipulator assembly 150.

The adapter 8 can include one or more intermediate rotary drivers 309 configured to couple to a corresponding rotary drive output 302 on the end effector 22 such that rotation of the corresponding rotary drive output 302 drives (rotates) the intermediate rotary driver 304. The intermediate rotary drivers 309 are also configured to mate/couple with corresponding rotary input(s) 330a, 330b, 330c of the instrument handle 31 such that rotation of the intermediate rotary driver 309, such as in response to rotation of a rotary drive output 302, correspondingly drives (rotates) one of the rotary inputs 330a-c.

In some configurations, a sterile drape 301, such as a plastic sheet or the like, may be disposed between the end effector 22 and the adapter 8 to provide a sterile barrier between the robot arm 12c and the instrument handle 31.

The instrument handle 31 can include a housing 80 that includes a base 80a and a cover 80b removably coupled to the base 80a to enclose various internal components of the instrument handle 31. As referenced above, the instrument handle 31 may further include a plurality of drive inputs 330a-c extending through the base 80a of the housing 80. In the illustrated embodiment, the instrument handle 31 includes three drive inputs 330a-c, although other numbers of drive inputs can be included in other embodiments. The drive inputs 330a-c can be in fixed positions spaced apart along the base 80a of the instrument handle 31, which facilitates coupling the drive inputs 330a-c to either the corresponding drive outputs 302 of the end effector 22 or the corresponding intermediate rotary drivers 309 in situations where an adapter 8 is utilized. The handle 31 can include latching clips 719 or other latching features/means for physically coupling to a corresponding structure of the adapter 8 and/or end effector 22.

An assembly within the instrument handle 31, described in more detail below, allows the drive inputs 330a, 330b to be used to drive articulation of the scope 40, whereas the drive input 330c can be used to drive roll of the shaft 40. Each of the drive inputs 330a-c can be configured to engage with a corresponding drive output 302 on the end effector 22 (a first drive interface) or a corresponding intermediate rotary driver 309 of the adapter 8 (a second drive interface). For example, each drive input 330a-c can comprise a receptacle or recess configured to mate with either an intermediate rotary driver 309 or a rotary output 302 that is configured as a spline. Rotation of the drive inputs 330a-c controls various functionality of the instrument handle 31, as will be discussed in more detail below.

FIG. 4 shows a cross-sectional view of the scope 40 of the instrument handle 31 as taken along the indicated lines in FIG. 3. The scope 40 includes a wall 312 that defines one of more lumens 310a, 310b, 310c, 310d that can be spaced equidistantly (or non-equidistantly) apart around the wall 312 of the scope 40. The wall 312 further defines a working channel 400 through which tools, such as lithotripters, basketing devices, forceps, etc., can be introduced into a treatment site. The scope 40 can also include one or more pull wires 314a, 314b, 314c, 314d that can be slidably disposed in the lumens 310a-d or through the working channel 400. The scope 40 may further include force isolation tubes (not shown) that extend through the lumens 310a-d or the working channel and 400 and through which the pull wires 314a-d extend. The pull wires 314a-d can include one or more wires, cables, fibers, and/or flexible shafts and can be made of any suitable or desirable materials, such as metallic and non-metallic materials, including stainless steel, Kevlar, tungsten, carbon fiber, and the like. Although a particular number of lumens 310a-d and pull wires 314a-d are illustrated in the figures, any number of lumens and/or pull wires can be implemented.

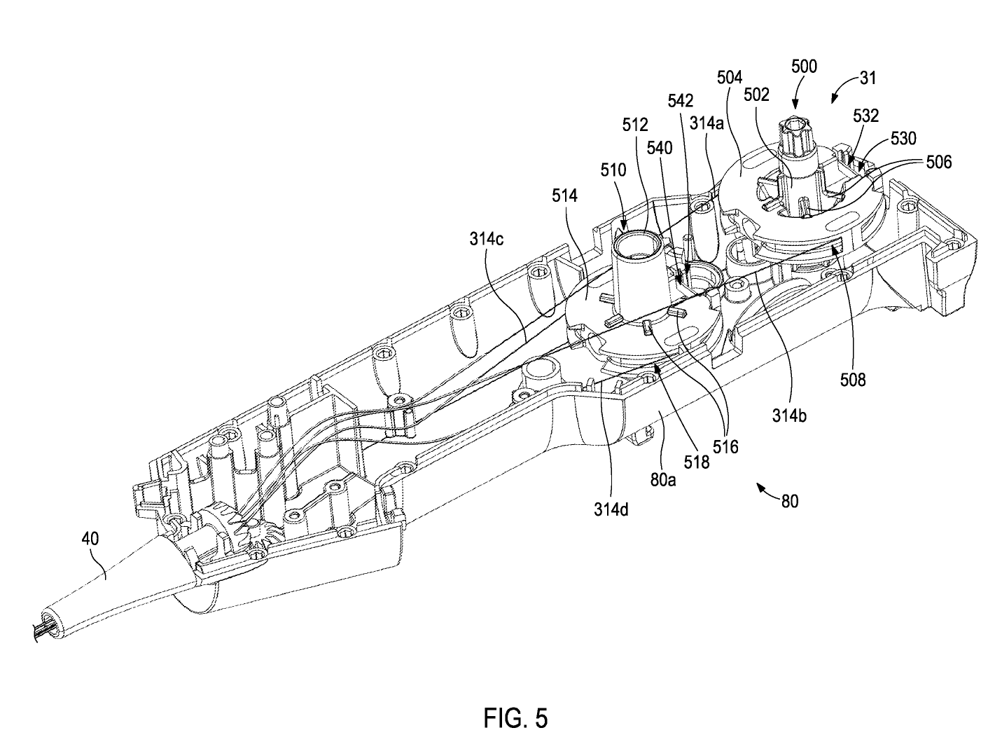

The pull wires 314a-d can be attached/extend to the distal section of the scope 40. At a proximal side, the pull wires 314a-d can be coupled to articulation drives 500, 510 (FIG. 5) mounted in the housing 80 of the instrument handle 31 that are configured to control articulation of the scope 40, as will be described in more detail below.

FIG. 5 shows an isometric view of the instrument handle 31 in accordance with at least one aspect of the present disclosure. As shown, the cover 80b of the housing 80 is omitted to allow viewing of various internal components of the instrument handle 31.

As illustrated, the instrument handle 31 includes a first and second articulation drives 500, 510 configured to control articulation of the scope 40. More specifically, the first articulation drive 500 may include a capstan 502 and a pulley 504. The capstan 502 and pulley 504 may be a unitary structure, or may be separate components coupled together. The capstan 502 may form part of or extend from the first drive input 330a (FIG. 3) such that rotation of the first drive input 330a (via actuation of a corresponding drive output 302 or intermediate rotary driver 309 of FIG. 3) correspondingly causes the capstan 502 and the pulley 504 to rotate.

The pulley 504 defines a groove 508 to receive the pull wires 314a,b. More specifically, the first pull wire 314a may be routed through the groove 508 on a first lateral side of the pulley 504, and the second pull wire 314b may be routed through the groove 508 on a second lateral side of the pulley 504 opposite the first lateral side. The pull wires 314a,b are operably coupled to the pulley 504 such that rotation of the pulley 504 causes corresponding longitudinal movement of the pull wires 314a, 314b. More specifically, rotation of the pulley 504 in a first radial direction (e.g., clockwise, as viewed in FIG. 5) causes the pulley 504 to apply tension to (pull) the first pull wire 314a and release tension in (push to allow slack) the second pull wire 314b, thereby causing the scope 40 to articulate in a first direction (e.g., right). Similarly, rotation of the pulley 504 in a second radial direction opposite the first radial direction (e.g., counterclockwise), causes the pulley 504 to apply tension to (pull) the second pull wire 314b and release tension in (push to allow slack) the first pull wire 314a, thereby causing the scope 40 to articulate in a second direction opposite the first direction (e.g., left).

Similar to the first articulation drive 500, the second articulation drive 510 may include a capstan 512 and a pulley 514. The capstan 512 and pulley 514 may be a unitary structure, or may be separate components coupled together. The capstan 512 may form part of or extend from the second drive input 330b (FIG. 3) such that rotation of the second drive input 330b (via actuation of a corresponding drive output 302 or intermediate rotary driver 309 (FIG. 3) correspondingly rotates the capstan 512 and the pulley 514.

The pulley 514 defines a groove 518 to receive the third and fourth pull wires 314c,d. More specifically, the third pull wire 314c may be routed through the groove 518 on a first lateral side of the pulley 514 and the fourth pull wire 314d may be routed through the groove 518 on a second lateral side of the pulley 514 opposite the first lateral side. The pull wires 314c,d are operably coupled to the pulley 514 such that rotation of the pulley 504 causes corresponding movement of the pull wires 314c,d. More specifically, rotation of the pulley 514 in a first radial direction (e.g., clockwise, as viewed in FIG. 5), causes the pulley 514 to apply tension to (pull) the third pull wire 314c and release tension in (push to allow slack) the fourth pull wire 314d, thereby causing the scope 40 to articulate in a third direction (e.g., up). Similarly, rotation of the pulley 514 in a second radial direction opposite the first radial direction (e.g., counterclockwise, as viewed in FIG. 5), causes the pulley 514 to apply tension to (pull) the fourth pull wire 314d and release tension in (push to allow slack) the third pull wire 314c, thereby causing the scope 40 to articulate in a fourth direction opposite the third direction (e.g., down).

### Pull Wire Termination via Simultaneous Pre-tensioning

As discussed above, the pull wires 314a-d may be used to articulate the scope 40 using the pulleys 504, 514. To reduce backlash in the system, slack is typically taken out of the pull wires 314a-d as the pull wires 314a-d are received at the corresponding pulley 504, 514. Systems and methods for securing the pull wires 314a-d to the pulleys 504, 514 that are quick, cheap, and maintain a predefined tension in the pull wires 314a-d over the life of the instrument handle 31 are desired.

As referenced above, the pull wires 314a-d may be operably coupled to the pulleys 504, 514 such that rotation of the pulleys 504, 514 yields articulation of the scope 40. Each pulley 504, 514 defines a slot 530, 540 respectively, sized to receive a corresponding wire anchoring member or “wire anchor” 532, 542 therein. The wire anchoring member 532, 542 may comprise a sleeve or a ferrule designed to receive and capture (secure) corresponding pull wires 314a-d.

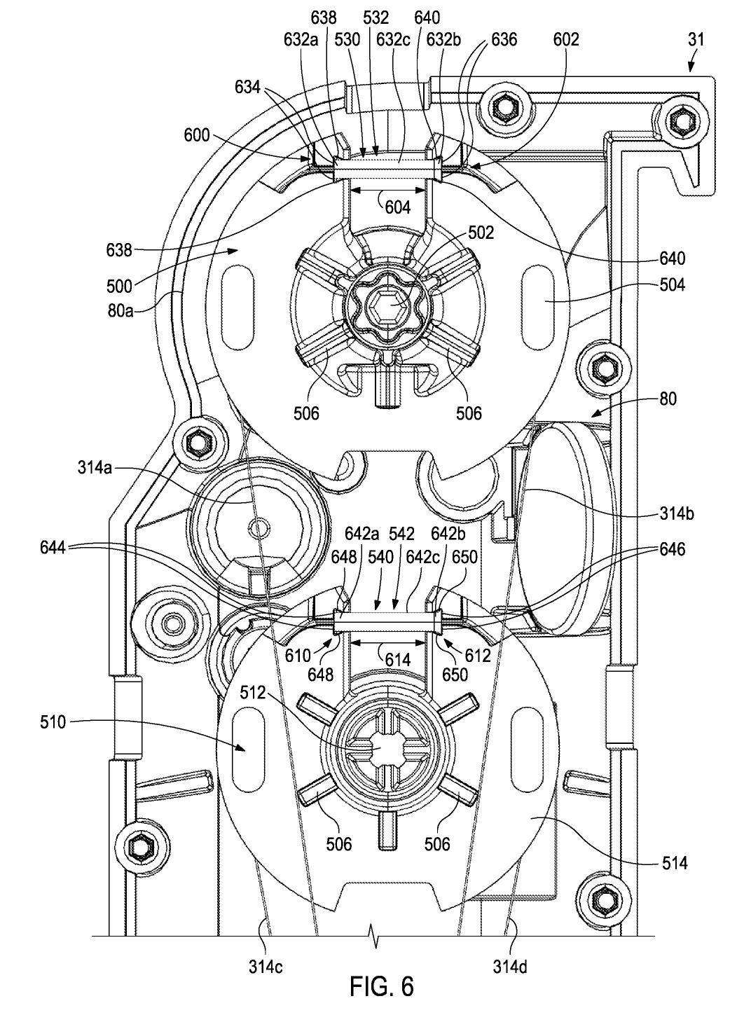

With reference now to FIG. 6, the wire anchoring members 532, 542 may comprise opposing first and second ends 632a, 642a and 632b, 642b, and define a channel (not shown) therethrough from the first end 632a, 642a to the second end 632b, 642b. The wire anchoring members 532, 542 may exhibit a first length that spans from the first end 632a, 642a to the second end 632b, 642b, respectively. In at least one embodiment, one or both of the ends 632a, 642a of either wire anchoring member 532, 542 may be flared radially outward.

The first pulley 504 may comprise a first support 600 to support the first end 632a of the wire anchoring member 532 and a second support 602 to support the second end 632b of the wire anchoring member 532. The first pulley 504 may define a gap 604 that separates the first and second supports 600, 602 from one another such that a center portion 632c of the wire anchoring member 532 is suspended when the first and second ends 632a,b are supported by the first and second supports 600, 602, respectively.

The first support 600 may provide first sidewalls 634 that co-operatively define a gap therebetween to receive the first pull wire 314a from the groove 508, and the second support 602 may similarly provide second sidewalls 636 that co-operatively define a gap therebetween to receive the second pull wire 314b from the groove 508. The first and second sidewalls 634, 636 may be separated by a second length that is the same, or substantially the same, as the first length of the wire anchoring member 532. Accordingly, when positioned in the slot 530, the first and second sidewalls 634, 636 may restrain the wire anchoring member 532.

The first and second supports 600, 602 may further provide third and fourth sidewalls 638, 640, respectively, that extend from the first and second sidewalls 634, 636, respectively, toward one another. Accordingly, the third and fourth sidewalls 638, 640 converge toward each other and may be sized to receive corresponding flared ends 632a, 632b of the wire anchoring member 638. In at least one embodiment, when the wire anchoring member 532 is received within the slot 530, the first and second sidewalls 634, 636 engage opposing sides of the wire anchoring member 532, thereby restraining the wire anchoring member 532 in the slot 530.

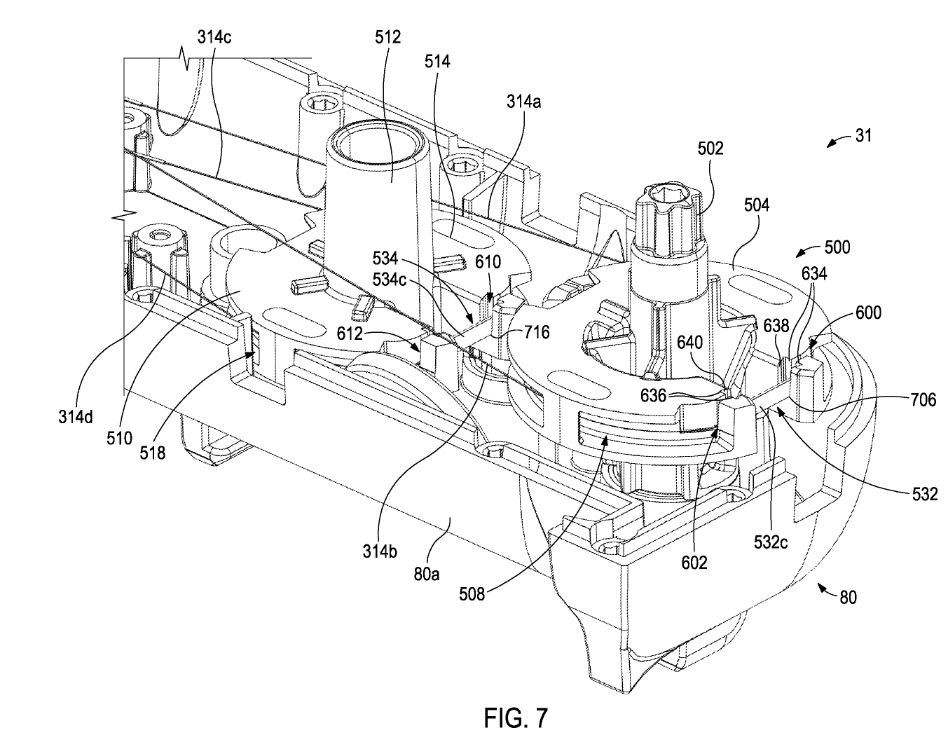

In some embodiments, the first wire anchoring member 532 may be received within the first slot 530 via an interference or friction fit, and thereby both vertically and laterally restraining the wire anchoring member 532. The first and second supports 600, 602 may further comprise a base 706 (base 706 of first support 600 can be seen in FIG. 7, but base 706 of second support 602 is occluded from view) upon which the first wire anchoring member 532 may be seated within the slot 530. In at least one embodiment, the first wire anchoring member 532 may be secured within the first slot 530 using, for example, an adhesive, swaging, welding, brazing, a mechanical fastener, or any combination thereof. Accordingly, the geometry of the first slot 530 and the optional adhesive may co-operatively maintain a position of the first wire anchoring member 532 within the slot 530 in six-degrees of freedom (e.g., x, y, z, rx, ry, and rz directions).

Similar to the first pulley 504, the second pulley 514 may include a first support 610 to support the first end 642a of the second wire anchoring member 542, and a second support 612 to support the second end 642b of the second wire anchoring member 542. The pulley 514 may define a gap 614 that separates the first and second supports 610, 612 from one another such that a center portion 642c of the wire anchoring member 542 is suspended when the first and second ends 642a,b are supported by the first and second supports 610, 612, respectively.

The first support 610 may comprise first sidewalls 644 that co-operatively define a gap therebetween to receive the third pull wire 314c from the groove 518. The second support 612 may comprise second sidewalls 646 that co-operatively define a gap therebetween to receive the fourth pull wire 314d from the groove 518. A second distance may be defined between the first and second sidewalls 644, 646 that is the same, or substantially the same, as the first distance defined between the first end 642a and the second end 642b of the wire anchoring member 542. Accordingly, when positioned in the slot 540, the first and second sidewalls 644, 646 may axially restrain the wire anchoring member 542.

The first and second supports 610, 612 may further comprise third and fourth sidewalls 648, 650, respectively, that extend from the first and second sidewalls 644, 646, respectively, and toward one another. In at least one embodiment, when second the wire anchoring member 542 is received within the second slot 532, the third and fourth second sidewalls 648, 650 engage opposing front and back sides of the wire anchoring member 542, thereby restraining the wire anchoring member 542 in the slot 540.

In some embodiments, second wire anchoring member 542 may be received within the slot 540 via an interference or friction fit, and thereby both vertically and laterally restraining the second wire anchoring member 542. The first and second supports 610, 612 may further provide a base 716 (base 716 of first support 610 can be seen in FIG. 7, but base 716 of second support 612 is occluded from view) upon which the anchoring member 542 may be seated within the slot 540. In at least one embodiment, the wire anchoring member 542 may be secured within the slot 540 using, for example, an adhesive, welding, brazing, or a mechanical fastener. Accordingly, the geometry of the slot 540 and the optional adhesive may co-operatively maintain a position of the wire anchoring member 542 within the slot 540 in three-degrees of freedom (e.g., x, y, and z directions).

With reference to FIGS. 6 and 7, during assembly of the first articulation drive 500, the wire anchoring member 532 may be positioned (seated) within the slot 530 against the bases 706 of the first and second supports 600, 602. Once the wire anchoring member 532 is received within the slot 530, the first pull wire 314a may be routed (fed) along the first lateral side of the pulley 504, through the groove 508 between the first sidewalls 634, into the first end 532a of the wire anchoring member 532, through the channel, and out the second end 532b of the wire anchoring member 532. Similarly, the second pull wire 314b may be routed (fed) along the second lateral side of the pulley 504, through the groove 508 between the second sidewalls 636, into the second end 532b of the wire anchoring member 532, through the channel, and out the first end 532a of the wire anchoring member 532.

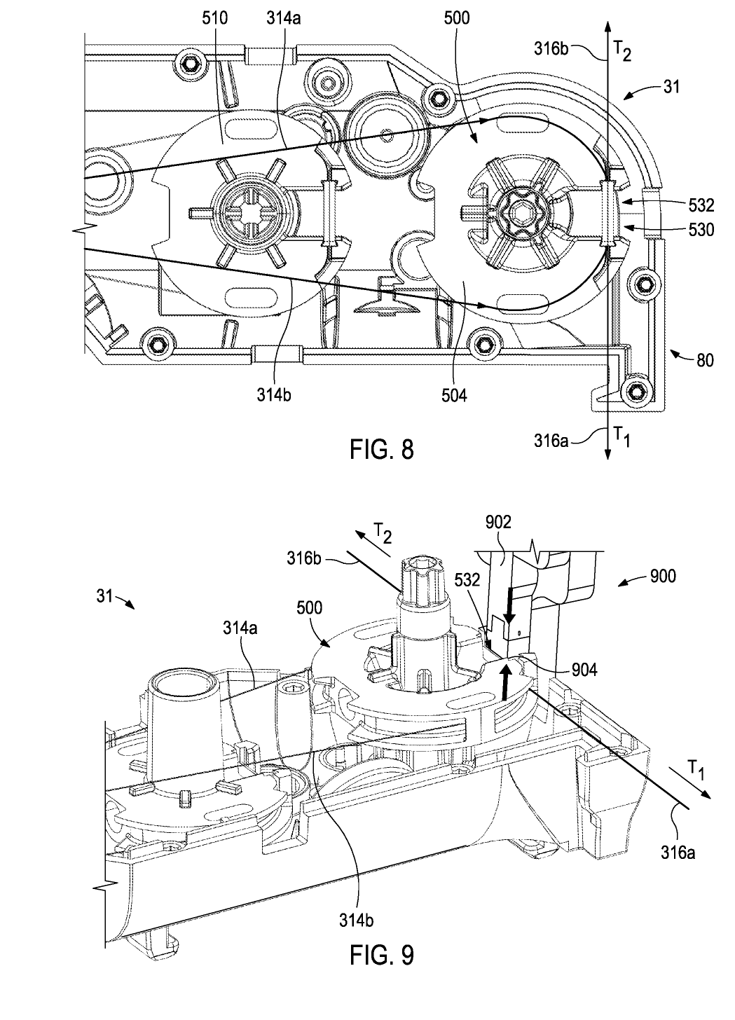

With reference now to FIGS. 8 and 9, with the first and second pull wires 314a,b fed through and exiting opposing ends 532a,b of the wire anchoring member 532, a predetermined (predefined) tension may be applied to each pull wire 314a,b to eliminate slack therefrom. For instance, a first weight (not shown) may be attached to a free end 316a of the first pull wire 314a to apply a predefined tension T₁ to the first pull wire 314a. Similarly, a second weight (not shown) may be attached to a free end 316b of the second pull wire 314b to apply a predefined tension T₂ to the second pull wire 314b.

With the tensions T₁, T₂ applied to the first and second pull wires 314a,b, a compression tool 900 may be arranged within the gap 604 (FIG. 6) and actuated to compress or crimp the wire anchoring member 532 onto the first and second pull wires 314a,b, thereby operably coupling and joining the pull wires 314a,b to the pulley 504 via the wire anchoring member 532. Compressing (crimping) the wire anchoring member 532 onto the pull wires 314a,b allows the wire anchoring member 532 to maintain the predefined tension T₁, T₂ within the pull wires 314a,b. In at least one embodiment, compressing (crimping) the wire anchoring member 532 onto the pull wires 314a,b plastically deforms the anchoring member 532 and thereby creates an interference fit between anchoring member 532 and the pulley 504.

The compression tool 900 may comprise any type of tool or device capable of crushing the wire anchoring member 532 and thereby capturing the pull wires 314a,b therein. In one embodiment, the compression tool 900 may comprise a pneumatic compression tool with opposing first and second jaws 902, 904 that co-operatively compress (crimp) the wire anchoring member 532. After the wire anchoring member 532 is compressed (crimped), the free ends 316a,b of the pull wires 314a,b may then be cut at the exit points of the wire anchoring member 532, thereby removing excess material.

While the above-provided discussion is regarding assembly of the first articulation drive 500, it should be understood that assembly of the second articulation drive 510 may be performed in a substantially similar manner.

Accordingly, the foregoing wire anchoring members 532, 542 and assembly thereof enable the pull wires 314a-d to be coupled to the pulleys 504, 514 in a manner that is quick, cheap, and maintains a predefined tension over the life of the instrument handle 31.

### Zero-Backlash Coupling for Ureteroscopy

Referring again to FIG. 3, each drive input 330a-c is configured to engage with a corresponding drive output 302 on the end effector 22 (a first drive interface) or a corresponding intermediate rotary driver 309 of the adapter 8 (a second drive interface). For example, each drive input 330a-c can comprise a receptacle or recess configured to mate with either an intermediate rotary driver 309 of the first drive interface or a rotary output 302 of the second drive interface. In such embodiments, the mated interface may comprise a splined male-female configuration.

Due to mechanical tolerances, rotation of the drive outputs 302 and/or the intermediate rotary drivers 309 may not yield corresponding (e.g., 1:1) movement of the drive inputs 330a-c. For example, gaps may be present between the spline and the corresponding receptacle of the drive inputs 330a-c such that there is a period of time where the drive outputs 302 and/or the intermediate rotary drivers 309 rotate, but the corresponding drive inputs 330a-c do not engage to rotate. This may cause a user to believe the instrument handle 31 is not properly functioning. As a result, the user may apply additional rotatory motion to the drive inputs 330a-c, which may cause the scope 40 to overshoot its intended position, such as within the urinary tract (63, 60, 65; FIG. 1), causing frustration in the user and/or potentially causing harm to the patient 7 (FIG. 1). Accordingly, systems and methods for eliminating backlash between these rotary components are desired.

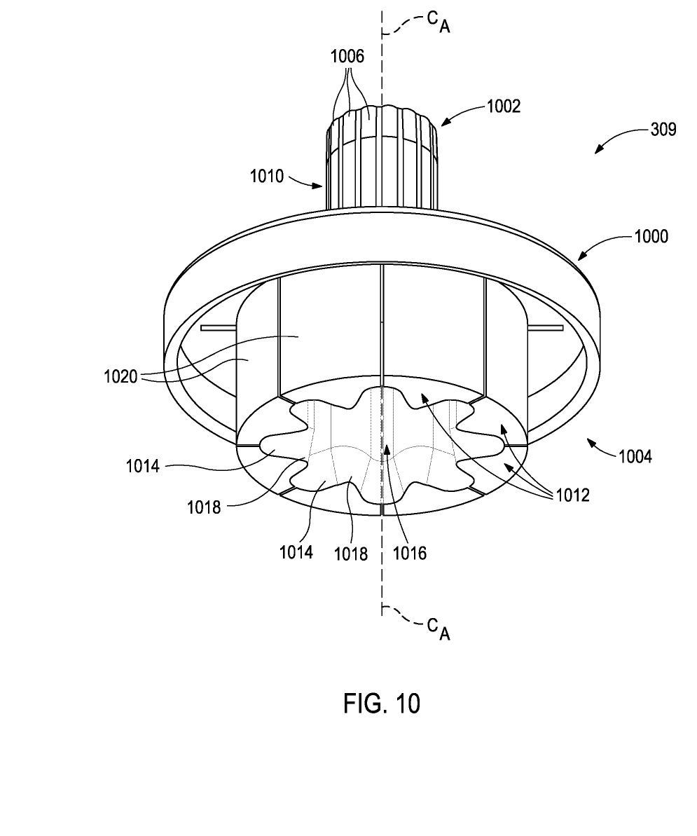

FIG. 10 is an improved intermediate rotary driver 309 of the adapter 8 (FIG. 3), in accordance with at least one aspect of the present disclosure. The intermediate rotary driver 309 may comprise a body 1000, a shaft 1002 extending from the body 1000 in a first direction, and a base 1004 extending from the body 1000 in a second direction opposite the first direction. The shaft 1002 may define a central axis C_{A} and may provide a castellated or splined interface. In particular, the shaft 1002 may define or otherwise provide a plurality of teeth 1006 circumferentially disposed about the central axis C_{A}.

The intermediate rotary driver 309 may be provided with one or more anti-backlash components configured to reduce backlash that may occur during rotation of the rotary output 302 (FIG. 3) and/or the intermediate rotary driver 309. For instance, the shaft 1002 may include, or be coated with, a first anti-backlash component 1010, which may be a material coated onto the teeth 1006 to eliminate, or at least substantially reduce, backlash between the intermediate rotary driver 309 and the drive inputs 330a-c (FIG. 3). The material of the anti-backlash component 1010 may be a compressible or compliant material, such as foam, rubber, gel, clay, a polymer (e.g., polyethylene, polypropylene foam, a sponge, a thermoplastic polymer, such as low-density polyethylene (LDPE) or polylactic acid (PLA)), an aerogel, wax, or any combination thereof.

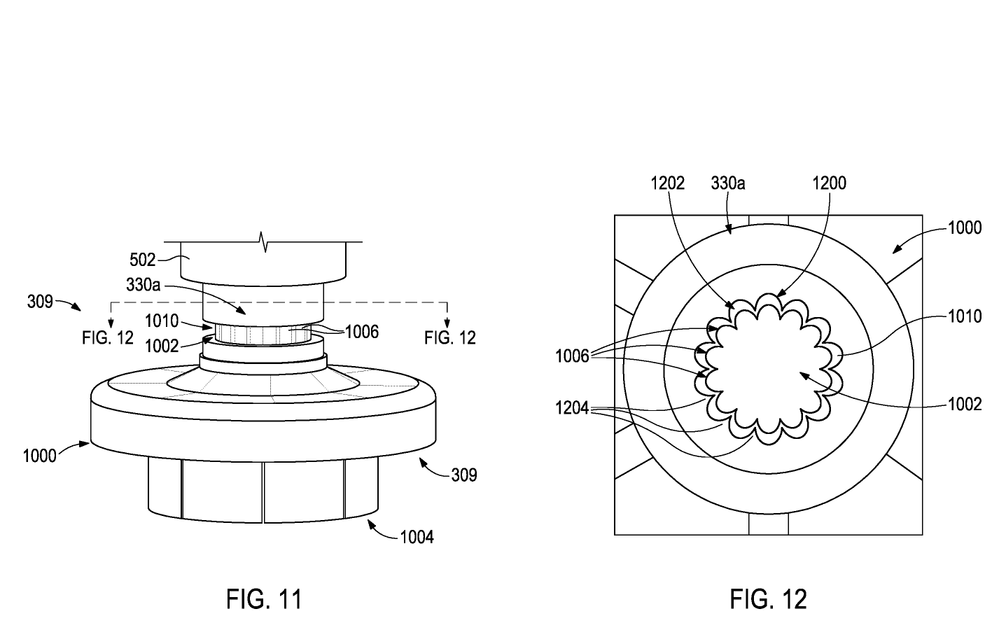

FIG. 11 shows the intermediate rotary driver 309 of FIG. 10 operably coupled to the capstan 502 (FIG. 5) of the articulation drive 500 (FIG. 5), and FIG. 12 is a top-down cross-sectional view of FIG. 12 as taken along the lines indicated in FIG. 11, in accordance with at least one aspect of the present disclosure. As shown in FIG. 12, the drive interface 330a of the capstan 502 may include a surface 1200 that defines a recess 1202 to receive the shaft 1002 of the intermediate rotary driver 309 and that provides teeth 1204 sized to received and otherwise intermesh with the teeth 1006 of the intermediate rotary driver 309. A radial gap defining a first volume may be defined between the shaft 1002 / teeth 1006 and the surface 1200 of the capstan 502, and the first anti-backlash component 1010 may include a second volume that is the same, or greater than the first volume. Accordingly, as the shaft 1002 is inserted into the recess 1202, the first anti-backlash component 1010 may be compressed between the shaft 1002 / teeth 1006 and the surface 1200 to fill (eliminate) the radial gap defined therebetween. As the shaft 1002 rotates, the first anti-backlash component 1010 may engage the surface 1200 of the drive interface 330a to rotate the capstan 502.

In the alternative, or in combination with the above, the drive interface 330a of the capstan 502 may be provided with one or more anti-backlash components. For example, the surface 1200 of the drive interface 330a may include, or be coated with, an anti-backlash component, like anti-backlash component 1010, which may be a material coated onto the teeth 1204 to eliminate, or at least substantially reduce, backlash between the intermediate rotary driver 309 and the drive input 330a.

The material of the first anti-backlash component 1010 may be selected with a stiffness that allows the shaft 1002 to be inserted into the recess 1202 with manual force from a user, but provides a sufficient stiffness to transfer torque from the shaft 1002 / teeth 1006 to the surface 1200 of the drive interface 330a.

With reference again to FIG. 10, the base 1004 may comprise, or otherwise define, a plurality of second anti-backlash components 1012 each including an inner surface 1014 that co-operatively define a recess 1016 with a first maximum diameter to receive a rotary output 302 (FIG. 3) of the end effector 22 (FIG. 3). The inner surfaces 1014 may each define a corresponding tooth 1018. The second anti-backlash components 1012 may each further define an outer surface 1020 to co-operatively define a circular, or substantially circular, profile of the base 1004. The second anti-backlash components 1012 may be columns, segments, or teeth that extend from the body 1000.

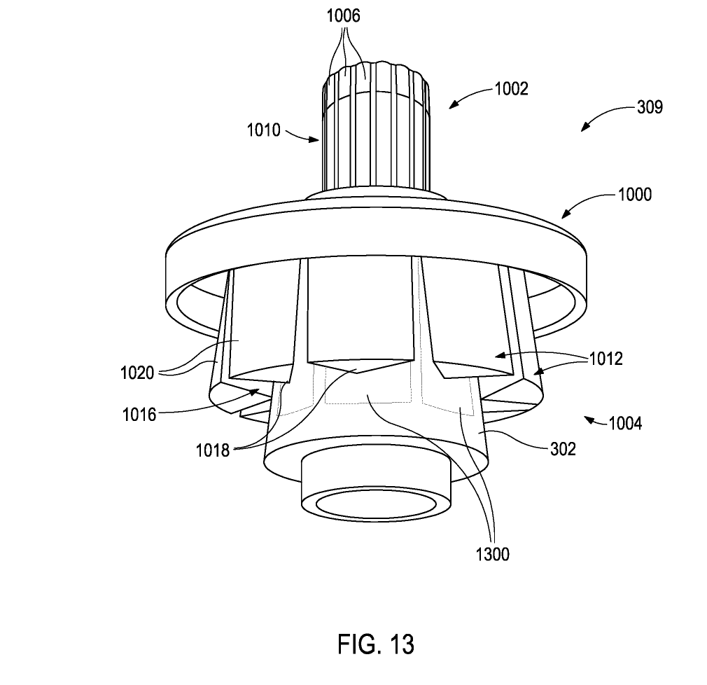

Each second anti-backlash component 1012 may be pivotably coupled to the base 1004, such as with a living hinge, that allows the second anti-backlash components 1012 to rotate (pivot or flex) relative to the body 1000 between a first position (FIG. 10), in which a first distance is defined between each tooth 1018 and the central axis C_{A}, and a second position (FIG. 13), in which a second distance greater than the first distance is defined between each tooth 1018 and the central axis C_{A}. The living hinge may naturally bias the second anti-backlash components 1012 toward the first position.

FIG. 13 shows a rotary output 302 of the end effector 22 (FIG. 3) coupled with the intermediate rotary driver 309, in accordance with at least one aspect of the present disclosure. As shown, when the rotary output 302 is inserted into the recess 1016, the rotary output 302 expands to the second diameter, such as being cammed (splayed) outwardly and away from one another toward their respective second positions. The second anti-backlash components 1012 may be naturally biased toward their respective first positions, and may thus facilitate a secure engagement with the rotary output 302. More specifically, the teeth 1018 of the second anti-backlash components 1012 may be naturally biased to intermesh with teeth 1300 (FIG. 13) of the rotary output 302, thereby eliminating any radial gap between the rotary output 302 and the intermediate rotary driver 309, which may eliminate, or substantially reduce, backlash therebetween.

The second anti-backlash components 1012 may be made of a material with a stiffness that enables the anti-backlash components 1012 to maintain, or at least substantially maintain, engagement with the rotary output 302 as the rotary output 302 rotates due to torque applied to the teeth 1018 from the teeth 1300. In the alternative, or in combination therewith, a retainer (not shown), such as a snap ring, a rubber band, or a collar, as examples, may be positioned around the base 1004 to engage each of the outer surfaces 1020 to prevent the teeth 1018 from demeshing from the teeth 1300.

In the alternative, or in combination with the above, the rotary output 302 and the teeth 1300 may also be coated with an anti-backlash component, like the first anti-backlash component 1010 (FIG. 10). In the alternative, or in combination with the above, the drive interface 330a (FIG. 11) of the capstan 502 (FIG. 11) may include a plurality of anti-backlash components, like second anti-backlash components 1012, that co-operatively define a recess with a first diameter and the shaft 1002 (FIG. 10) may define a second diameter greater than the first diameter, thereby providing a similar interface between the intermediate rotary driver 309 and the rotary inputs 330a-c (FIG. 3) as is provided between the rotary output 302 and the intermediate rotary driver 309, as shown in FIG. 13.

Accordingly, the foregoing anti-backlash components eliminate backlash that may arise between the rotary output 302 and the intermediate rotary driver 309 and/or the capstan 502 and the intermediate rotary driver 309.

Embodiments disclosed herein include:
A. A surgical system comprising a housing, a scope extending from the housing, first and second wires operably coupled to the scope and extending into the housing, a pulley rotatably mounted within the housing and defining a slot extending between first and second supports of the pulley, and a wire anchor received within the slot and configured to receive the first and second wires, wherein joining the wire anchor to the first and second wires operably couples the first and second wires to the pulley.
B. A method comprising feeding a first wire through a wire anchor positioned in a slot of a pulley rotatably mounted within a housing of a surgical instrument, feeding a second wire through the wire anchor, and joining the wire anchor to the first and second wires to operably couple the first and second wires to the pulley.
C. A surgical system comprising a surgical instrument and a drive interface operably couplable with the surgical instrument. The surgical instrument comprises a housing, a scope extending from the housing, a wire operably coupled to the scope, a pulley within the housing and operably coupled to the wire, wherein the wire is to articulate the scope based on rotation of the pulley, and a capstan coupled to the pulley, wherein rotation of the pulley is based on rotation of the capstan. The drive interface comprises a rotary driver, wherein rotation of the capstan is based on rotation of the rotary driver, and wherein the rotary driver comprises an anti-backlash component to eliminate backlash from the rotary driver to the capstan.
D. A surgical system comprising a surgical instrument and an adapter removably couplable with the surgical instrument. The surgical instrument comprises a housing, a scope extending from the housing, a wire operably coupled to the scope, a pulley within the housing and operably coupled to the wire, wherein the wire is to articulate the scope based on rotation of the pulley, and a capstan coupled to the pulley, wherein rotation of the pulley is based on rotation of the capstan. The adapter comprises a rotary driver couplable with the capstan, and wherein the rotary driver comprises a compliant coating.
E. A surgical system comprising a surgical instrument and an adapter removably couplable with the surgical instrument. The surgical instrument comprises a housing, a scope extending from the housing, a wire operably coupled to the scope, a pulley within the housing and operably coupled to the wire, wherein the wire is to articulate the scope based on rotation of the pulley, and a capstan coupled to the pulley, wherein rotation of the pulley is based on rotation of the capstan. The adapter comprises a rotary driver couplable with the capstan, and wherein the rotary driver comprises segments that are rotatable relative to one another.

Each of embodiments A-E may have one or more of the following additional elements in any combination: Element 1: wherein the wire anchor includes opposing first and second ends, and wherein the first and second supports laterally and longitudinally restrain the first and second ends. Element 2: wherein at least one of the first and second ends of the wire anchor is flared radially outward. Element 3: wherein at least one of the first and second supports comprises opposing sidewalls that converge toward one another. Element 4: wherein the first wire is routed on a first side of the pulley and extends into the first end and the second wire is routed on a second side of the pulley and extends into the second end. Element 5: further comprising an adhesive applied to secure the wire anchor to the first and second supports. Element 6: wherein the wire anchor comprises a ferrule. Element 7: wherein joining the wire anchor to the first and second wires generates an interference fit between the wire anchor and the pulley. Element 8: wherein the pulley is mounted to a capstan positioned within the housing, and wherein rotating the capstan correspondingly rotates the pulley and thereby moves the first and second wires. Element 9: wherein joining the wire anchor comprises crimping the wire anchor onto the first and second wires in the slot. Element 10: wherein joining the wire anchor comprises crimping the wire anchor with a pneumatic tool. Element 11: wherein joining the wire anchor is preceded by applying tension to the first and second wires and joining the wire anchor while applying the tension. Element 12: wherein applying tension to the first and second wires comprises applying tension to the first wire with a first weight and applying tension to the second wire with a second weight. Element 13: further comprising routing the first wire along a first lateral side of the pulley to a first end of the wire anchor and routing the second wire along a second lateral side of the pulley opposite the first lateral side to a second end of the wire anchor opposite the first end. Element 14: wherein the slot provides opposing first and second ends and feeding the first wire through the wire anchor is preceded by receiving the first end of the wire anchor at the first end of the slot and receiving the second end of the wire anchor at the second end of the slot. Element 15: wherein at least one end of the slot provides a sidewall that converges, and wherein at least one end of the wire anchor comprises a flared end, the method further comprising receiving the flared end within the sidewall that converges. Element 16: further comprising securing the wire anchor to the pulley with an adhesive. Element 17: further comprising operably coupling the first and second wires to a scope extending from the housing. Element 18: wherein joining the wire anchor onto the first and second wires further comprises plastically deforming the wire anchor and thereby creating an interference fit between wire anchor and the pulley. Element 19: wherein the anti-backlash component comprises a compliant coating. Element 20: wherein the rotary driver comprises a shaft providing teeth, and wherein the compliant coating is coated on the shaft and the teeth. Element 21: wherein the capstan includes a surface that defines a recess, a radial gap is defined between the teeth and the surface based on the shaft being received in the recess, and the compliant coating fills the radial gap. Element 22: wherein the radial gap defines a first volume, and wherein the compliant coating defines a second volume greater than the first volume. Element 23: wherein the rotary driver comprises a plurality of anti-backlash components comprising the anti-backlash component. Element 24: wherein the plurality of anti-backlash components comprises columns that are rotatable relative to one another. Element 25: wherein the columns co-operatively define a recess, and wherein the columns are rotatable away from one another based a rotary output being received in the recess. Element 26: wherein each column of the columns provides a tooth, and wherein the rotary output defines teeth to intermesh with the teeth of the columns. Element 27: further comprising an adapter removably couplable with the surgical instrument and a robotic arm, wherein the adapter comprises the drive interface. Element 28: wherein the rotary driver comprises a shaft providing teeth, and wherein the compliant coating is coated on the shaft and the teeth. Element 29: wherein the capstan includes a surface that defines a recess, a radial gap is defined between the teeth and the surface based on the shaft being received in the recess, and the compliant coating fills the radial gap. Element 30: wherein the radial gap defines a first volume, and wherein the compliant coating defines a second volume greater than the first volume. Element 31: wherein the rotary driver comprises segments that are rotatable relative to one another. Element 32: wherein the segments co-operatively define a recess, and wherein the segments are rotatable away from one another based a rotary output of a robotic arm being received in the recess. Element 33: wherein each segment of the segments provides a tooth, and wherein the rotary output defines teeth to intermesh with the teeth of the segments. Element 34: wherein the segments co-operatively define a recess, and wherein the segments are rotatable away from one another based a rotary output of a robotic arm being received in the recess. Element 35: wherein each segment of the segments provides a tooth, and wherein the rotary output defines teeth to intermesh with the teeth of the segments.

By way of non-limiting example, exemplary combinations applicable to A, B, C, D, and E include: Element 1 with Element 2; Element 1 with Elements 2 and 3; Element 1 with Element 2; Element 9 with Element 10; Element 11 with Element 12; Element 13 with Element 14; Element 19 with or more of Elements 20-22; Element 23 with one or more of Elements 24-26; Element 28 with one or both of Elements 29 and 30; Element 31 with one or more of Elements 32 and 33; Element 34 with Element 35.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A surgical system, comprising:
a housing;
a scope extending from the housing;
first and second wires operably coupled to the scope and extending into the housing;
a pulley rotatably mounted within the housing and defining a slot extending between first and second supports of the pulley; and
a wire anchor received within the slot and configured to receive the first and second wires,
wherein joining the wire anchor to the first and second wires operably couples the first and second wires to the pulley.

2. The surgical system of claim 1, wherein the wire anchor includes opposing first and second ends, and wherein the first and second supports laterally and longitudinally restrain the first and second ends.

3. The surgical system of claim 2, wherein at least one of the first and second ends of the wire anchor is flared radially outward.

4. The surgical system of claim 3, wherein at least one of the first and second supports comprises opposing sidewalls that converge toward one another.

5. The surgical system of claim 2, wherein:
the first wire is routed on a first side of the pulley and extends into the first end; and
the second wire is routed on a second side of the pulley and extends into the second end.

6. The surgical system of claim 1, further comprising an adhesive applied to secure the wire anchor to the first and second supports.

7. The surgical system of claim 1, wherein the wire anchor comprises a ferrule.

8. The surgical system of claim 1, wherein joining the wire anchor to the first and second wires generates an interference fit between the wire anchor and the pulley.

9. The surgical system of claim 1, wherein the pulley is mounted to a capstan positioned within the housing, and wherein rotating the capstan correspondingly rotates the pulley and thereby moves the first and second wires.

10. A method, comprising:
feeding a first wire through a wire anchor positioned in a slot of a pulley rotatably mounted within a housing of a surgical instrument;
feeding a second wire through the wire anchor; and
joining the wire anchor to the first and second wires to operably couple the first and second wires to the pulley.

11. The method of claim 10, wherein joining the wire anchor comprises crimping the wire anchor onto the first and second wires in the slot.

12. The method of claim 11, wherein joining the wire anchor comprises crimping the wire anchor with a pneumatic tool.

13. The method of claim 10, wherein joining the wire anchor is preceded by applying tension to the first and second wires and joining the wire anchor while applying the tension.

14. The method of claim 13, wherein applying tension to the first and second wires comprises:
applying tension to the first wire with a first weight; and
applying tension to the second wire with a second weight.

15. The method of claim 10, further comprising:
routing the first wire along a first lateral side of the pulley to a first end of the wire anchor; and
routing the second wire along a second lateral side of the pulley opposite the first lateral side to a second end of the wire anchor opposite the first end.

16. The method of claim 15, wherein the slot provides opposing first and second ends and feeding the first wire through the wire anchor is preceded by:
receiving the first end of the wire anchor at the first end of the slot; and
receiving the second end of the wire anchor at the second end of the slot.

17. The method of claim 10, wherein at least one end of the slot provides a sidewall that converges, and wherein at least one end of the wire anchor comprises a flared end, the method further comprising receiving the flared end within the sidewall that converges.

18. The method of claim 10, further comprising securing the wire anchor to the pulley with an adhesive.

19. The method of claim 10, further comprising operably coupling the first and second wires to a scope extending from the housing.

20. The method of claim 10, wherein joining the wire anchor onto the first and second wires further comprises plastically deforming the wire anchor and thereby creating an interference fit between wire anchor and the pulley.
